(19) United States
(12) Patent Application Publication
(10) Pub. No.: US 2020/0089010 A1
(43) Pub. Date: Mar. 19, 2020

(54) MEDICAL MAGNIFICATION DEVICE

(71) Applicant: Masafumi Koshiyama, Kyoto-shi (JP)

(72) Inventor: Masafumi KOSHIYAMA, Kyoto (JP)

(21) Appl. No.: 16/685,013

(22) Filed: Nov. 15, 2019

Related U.S. Application Data
(63) Continuation of application No. PCT/IB2018/000739, filed on Jul. 12, 2018.

(51) Int. Cl. G02B 27/02; G02B 27/01; A61B 17/42; G02B 7/08

(52) U.S. Cl. CPC G02B 27/027; G02B 7/08; A61B 17/42; G02B 27/017

(57) ABSTRACT

A medical magnification device, which is an example of an embodiment, comprises: a device body which has a face pad that comes into contact with the face of a user, and which covers the front of the left and right eyes of the user; a belt for fixing the device body to the head of the user; an imaging unit which has a zoom lens provided to the front of the device body, and which performs digital conversion on a magnified image obtained by the zoom lens; and a pair of monitors.

### CROSS REFERENCE TO RELATED APPLICATIONS

This application is the bypass continuation of PCT/IB2018/000739, filed on Jul. 12, 2018, which claims priority under 35 U.S.C. § 119(a) and 35 U.S.C. § 365(b) to Japanese Patent Application No. 2017-097097, filed May 16, 2017, the contents of which are incorporated herein by reference in their entirety.

### TECHNICAL FIELD

The present invention relates to a medical magnifier device, and particularly to a medical magnifier device suitable for a medical examination in obstetrics and gynecology.

### BACKGROUND ART

Medical magnifier devices such as microscopes and loupes are widely used in a medical examination, a treatment, and surgery by doctors and dentists. In general, since the performance required for the magnifier device varies depending on medical departments, use purposes, and the like, many dedicated devices specialized for respective medical departments, respective use purposes, and the like have been developed. For example, a dedicated magnifier device called a colposcope (uterovaginal magnifier) is used in the obstetrics and gynecology. The colposcope is mainly used for a thorough checkup of uterine cancer screening.

Patent Literature 1 proposes a colposcope in which a mirror body can move smoothly and rapidly. The colposcope disclosed in Patent Literature 1 includes a mirror body, a base portion that determines a position of the device as a whole, a vertical movement arm that moves the mirror body in a vertical direction, and a horizontal movement arm that moves the mirror body in a horizontal direction, and the horizontal movement arm includes a parallelogram link mechanism.

### CITATION LIST

### Patent Literature

Patent Literature 1: JP H08-140932 A

### SUMMARY

### Technical Problem

By the way, it is important that the medical magnifier device is compact and easy to use, as well as being able to clearly observe the affected area with a desired size. However, colposcopes for obstetrics and gynecology and microscopes for surgery are large and not easy to use, and are required for further improvement.

For example, the colposcope disclosed in Patent Literature 1 is assumed to be able to move the mirror body smoothly and rapidly as compared with the conventional colposcope, but there is still much room for improvement. The conventional colposcope including the technique of Patent Literature 1 can be moved using the caster of the base portion, but it is not preferable to force a doctor to perform such movement work. In the conventional colposcope, the base portion may be unavoidably moved even in a case of focusing on the affected area. Furthermore, since the arm exists between the doctor and the patient to support the mirror body, such an arm becomes an obstacle at the time of medical examination and treatment. In addition, a large colposcope may be difficult to place in position.

### Solution to Problem

A medical magnifier device according to an aspect of the present invention is a medical magnifier device of head-mounted type, and includes: a device body that includes a face pad coming into contact with a face of a user and covers left and right eyes of the user; a fixing member used to fix the device body to a head of the user; an imaging unit that includes a zoom lens attached to in front of the device body and performs digital conversion on a magnified image acquired through the zoom lens; and at least one monitor that is provided in front of the left and right eyes of the user at the inside of the device body and display the magnified image digitally converted by the imaging unit.

### Advantageous Effects of Invention

According to an aspect of the present invention, it is possible to provide medical magnifier device that is compact and easy to use as well as being able to clearly observe an affected area with a desired size.

### BRIEF DESCRIPTION OF DRAWINGS

FIG. 1 is a perspective view of a medical magnifier device which is an example of an embodiment.

FIG. 2 is a perspective view of the medical magnifier device, which is an example of the embodiment, as viewed from the inside of a device body.

FIG. 3 is a cross-sectional view of the device body which is an example of the embodiment.

FIG. 4 is a view illustrating a mounted state of the medical magnifier device which is an example of the embodiment.

FIG. 5 is a view illustrating a state of performing a medical examination using the medical magnifier device which is an example of the embodiment.

FIG. 6 is a view illustrating a state of performing surgery using the medical magnifier device which is an example of the embodiment.

FIG. 7 is a perspective view of a medical magnifier device which is another example of the embodiment.

### DESCRIPTION OF EMBODIMENTS

An example of an embodiment of a medical magnifier device according to the present invention will be described below in detail with reference to the drawings. The reference drawings in the embodiment are schematically described, and specific dimensions and the like of respective components should be determined in view of the following description. In addition, it is assumed from the beginning that configurations of several embodiments described below are combined appropriately.

A medical magnifier device 10 or 10X is illustrated below as an embodiment, but a medical magnifier device according to the present invention is not limited thereto. In addition, examples of use of the medical magnifier device 10 or 10X include a medical examination and surgery in obstetrics and gynecology, but the use of the medical magnifier device 10 or 10X is not limited thereto. The medical magnifier device 10 or 10X can also be used for medical examinations and treatments in various medical departments, for example, in dermatology, cardiac surgery, orthopedics, oral surgery/dentistry, and otolaryngology.

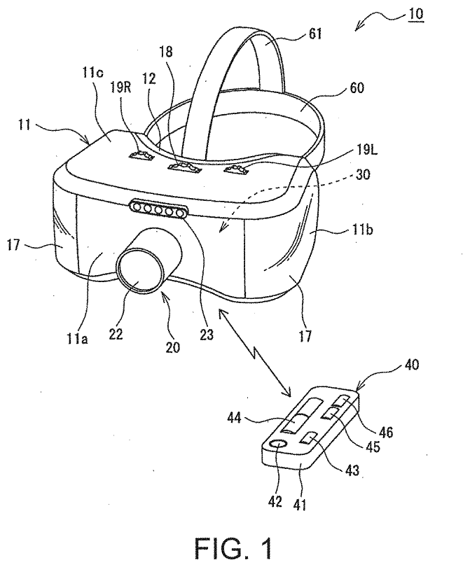

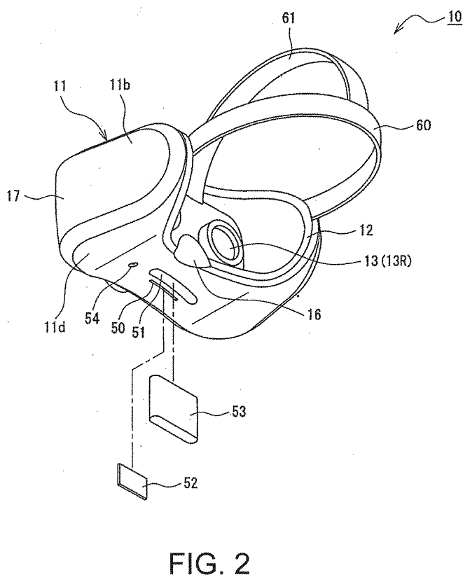

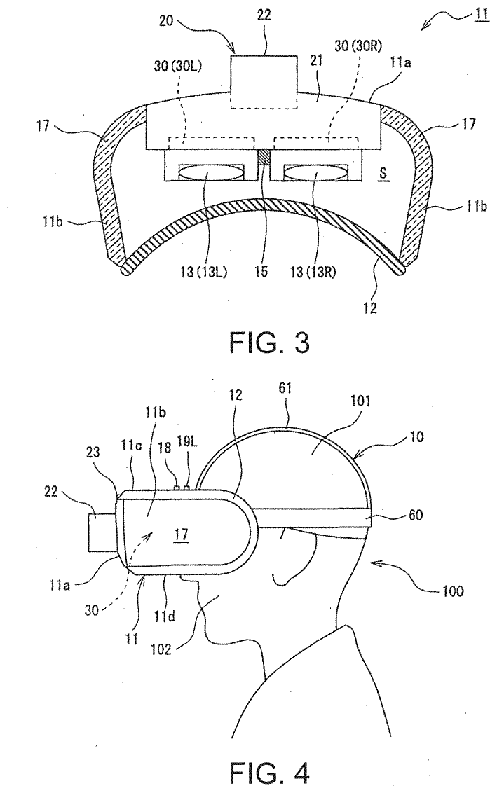

FIG. 1 is a perspective view of the medical magnifier device 10 that is an example of an embodiment. FIG. 2 is a view illustrating a device body 11 of the medical magnifier device 10 as viewed from the inside, and FIG. 3 is a cross-sectional view of the device body 11. FIG. 4 is a view illustrating a mounted state of the medical magnifier device 10. For convenience of explanation in this specification, a direction along a width direction of a face in the mounted state of the medical magnifier device 10 is referred to as “lateral direction or left-right direction” of the device body 11, and a direction along a length direction of the face is referred to as “up-down direction” of the device body 11.

As illustrated in FIG. 1 to FIG. 4, the medical magnifier device 10 is a head-mounted magnifier device including: a device body 11 that includes a face pad 12 coming into contact with a face 102 of a user 100 and covers left and right eyes of the user 100; and a fixing member used to fix the device body 11 to a head 101 of the user 100. In the present embodiment, the fixing member includes a belt 60 that is attached to left and right ends of the device body 11. The belt 60 extends from the left and right ends of the device body 11 to the rear of the head 101 and comes into contact with the back of the head, thereby the medical magnifier device 10 can be mounted on the head. Furthermore, the belt 60 may have a length adjustment function.

The medical magnifier device 10 may include an auxiliary belt 61 that serves as the fixing member and comes into contact with the top of the head. The auxiliary belt 61 extends from an upper end of the device body 11 toward the back of the head over the top of the head and is coupled to the belt 60 on the back of the head. In particular, when the weight of the device body 11 is large, a mounting property of the medical magnifier device 10 on the head is improved by the provision of auxiliary belt 61.

The medical magnifier device 10 includes: an imaging unit 20 that includes a zoom lens 22 provided in front of the device body 11 and performs digital conversion on a magnified image acquired through the zoom lens 22; and at least one monitor that displays the magnified image digitally converted by the imaging unit 20. The medical magnifier device 10 includes a pair of monitors 30 which are provided in front of the left and right eyes of the user 100 at the inside of the device body 11, as the at least one monitor. The medical magnifier device 10 includes a right-eye monitor 30R and a left-eye monitor 30L as the monitors 30.

The medical magnifier device 10 is not a device that is supported by the nose and ears as in eyeglasses, but is a goggle-type magnifier device in which the device body 11 is held in front of the eyes by the face pad 12 and the fixing member. Since the device body 11 firmly fits on the face 102 and is stably held in front of the eyes, a medical practice such as a medical examination or a treatment can be performed smoothly, and the user hardly get tired even after long time use. Moreover, the device body 11 covers around the eyes, and thus the visibility of the monitor 30 is improved. Furthermore, the medical magnifier device 10 has various advantages as compared to a colposcope used in obstetrics and gynecology. The description will be given below with regard to this.

The device body 11 is a portion on which the imaging unit 20 and the monitor 30 are mounted and to which the belt 60 and the auxiliary belt 61 are attached. The device body 11 is large enough to cover around the both eyes of the user 100, for example, has a vertical length sufficient to cover from a lower part of a forehead to an upper part of a cheek and a lateral length that is the same width as that of the face. In addition, the device body 11 has a thickness to a certain extent even in a front-rear direction so as to secure a mounting space for the imaging unit 20 and the monitor 30. The device body 11 is in a state of protruding forward about 50 mm to 150 mm from the face 102 of the user 100, for example.

The device body 11 includes a front surface portion 11a to which the zoom lens 22 is attached. Moreover, the device body 11 preferably includes a lateral surface portion 11b that is formed to extend backwardly from both left and right ends of the front surface portion 11a and to cover up to the vicinity of temples of the user 100. When the device body 11 is mounted to cover a lateral side of the face, a good fit feeling can be obtained. The front surface portion 11a may be formed substantially flat in the up-down direction and the lateral direction, and may be gently curved forward. The device body 11 is curved outward from boundary portions between the front surface portion 11a and the respective lateral surface portions 11b, and the boundary portion is rounded, but may have an angular shape with an edge.

In addition, the device body 11 preferably includes an upper surface portion 11c and a lower surface portion 11d that are formed to extend backwardly from both upper and lower ends of the front surface portion 11a and to cover upper and lower sides of eyes, respectively. In particular, when the upper surface portion 11c is provided, the visibility of the monitor 30 is improved and a good fit feeling is obtained. The device body 11 is preferably made of an opaque material except for a window portion 17 to be described below, and is suitably made of a resin in consideration of lightness, workability, and the like.

The device body 11 includes the face pad 12 coming into contact with the face 102 of the user 100 as described above. The face pad 12 is made of foam, elastomer, or rubber that is elastically deformed when coming into contact with the face 102. In the present embodiment, a recess portion 16 is formed in an inner part of the device body 11 facing the user 100 so as to avoid interference with the nose. The face pad 12 is provided at an inner end of the device body 11, excluding the part where the recess portion 16 is formed. The face pad 12 is provided along edges of the respective lateral surface portions 11b, the upper surface portion 11c, and the lower surface portion 11d of the device body 11, for example.

The zoom lens 22 is provided at a center, a left side, or a right side on the front surface portion 11a of the device body 11 so as to protrude forward. As illustrated in FIG. 3, a main body 21 of the imaging unit 20 and the monitor 30 are disposed in a space S formed between the front surface portion 11a and the inner end of the device body 11. In the present embodiment, the monitor 30 is installed on a surface (rear side) of the main body 21 facing the user 100. That is, the imaging unit 20 is formed integrally with the monitor 30.

A pair of eyepieces 13 may be disposed in the space S of the device body 11. The pair of eyepieces 13 includes a right-eye eyepiece 13R and a left-eye eyepiece 13L. In an example illustrated in FIG. 1, an inter-eyepiece distance adjusting portion 18 is provided on the upper surface portion 11c of the device body 11 to adjust a distance between the eyepieces 13R and 13L. Further, eyepiece focus adjusting portions 19R and 19L are provided on the upper surface portion 11c of the device body 11 to adjust a focus of the eyepieces 13R and 13L. On the upper surface portion 11c, the inter-eyepiece distance adjusting portion 18 is disposed at a position corresponding to a point between the left and right eyes of the user 100, and the eyepiece focus adjusting portions 19R and 19L are respectively disposed on the left and right sides of the upper surface portion 11c with the inter-eyepiece distance adjusting portion 18 interposed therebetween.

The inter-eyepiece distance adjusting portion 18 and the eyepiece focus adjusting portions 19R and 19L are configured by a dial installed in the device body 11. The dial is installed to partially protrude upwardly from the upper surface portion 11c. The user 100 can narrow or widen a gap between the eyepieces 13R and 13L according to a gap between user's eyes by turning the inter-eyepiece distance adjusting portion 18. In addition, the user 100 moves the eyepieces 13R and 13L in the front-rear direction by turning the eyepiece focus adjusting portions 19R and 19L, and thus can adjust the focus of each lens separately. Furthermore, the inter-eyepiece distance adjusting portion 18 and the eyepiece focus adjusting portions 19R and 19L may be provided as an operation switch of a remote controller 40 to be described below.

In the space S of the device body 11, a pair of monitors 30 are disposed in front of the pair of eyepieces 13. In a state where the medical magnifier device 10 is mounted, the eyepiece 13R and the monitor 30R are sequentially present in front of the right eye of the user 100, and the eyepiece 13L and the monitor 30L are sequentially present in front of the left eye. In the space S, a partition wall portion 15 is formed along the front-rear direction at the center in the lateral direction of the device body 11. The partition wall portion 15 isolates the monitors from each other such that the monitor 30R is not seen by the left eye and the monitor 30L is not seen by the right eye.

The device body 11 preferably includes the window portions 17 that are provided at both left and right sides with the imaging unit 20 interposed therebetween and allow the user 100 to visually recognize left and right sides. The device body 11 is preferably mounted so as to cover around both eyes from the viewpoint of the mounting property and the visibility improvement of the monitor 30, but when the entire device body 11 is made of an opaque material, surroundings may not be visible at all, which is inconvenient for medical practice. When the device body 11 includes the window portion 17, surrounding situations can be confirmed without removal of the device one by one, and the medical magnifier device 10 is easier to use.

The window portion 17 may be an opening portion formed in the device body 11, and is preferably made of a resin having good translucency such as an acrylic resin or polycarbonate. Moreover, the acrylic resin and the polycarbonate are also excellent in heat resistance, thereby being also suitable for sterilization treatment of the device body 11. The window portion 17 is preferably formed on each of the lateral surface portions 11b, and is provided in such a manner that a transparent resin material is applied to a part or all of each lateral surface portion 11b. On each of the lateral surface portion 11b, the window portions 17 are provided, for example, from the boundary portions with the curved front surface portion 11a or both the left and right sides of the front surface portion 11a to positions corresponding to points just beside the eyes. When the window portions 17 are provided to extend to both the left and right sides of the front surface portion 11a, the user 100 can visually recognize the left and right front in the state where the medical magnifier device 10 is mounted.

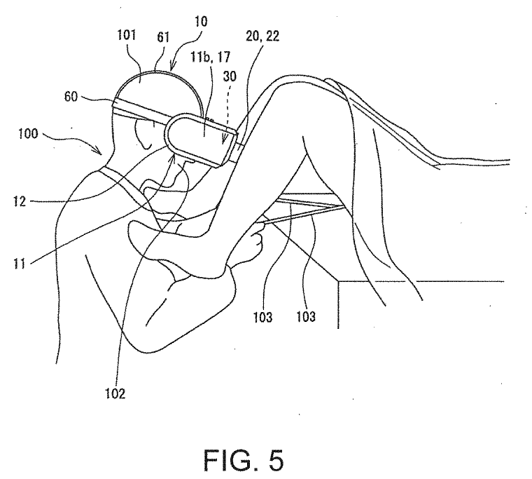

The window portions 17 are preferably formed below the positions at least corresponding to the points just beside the eyes. Since an instrument 103 (see FIG. 5 to be described below) or the like used for the medical examination is generally located below the eye level of the user 100, it is preferable that the window portion 17 is formed at least below the eye level. For example, on each of the lateral surface portions 11b of the device body 11, the window portion 17 is formed below the lower end of the fixing member such as the belt 60. In the present embodiment, in order to make the left and right visibility better, the window portions 17 are also formed greatly in the up-down direction of the device body 11 across the boundary portion of the lower surface portion 11d from the boundary portion of the upper surface portion 11c so as to be located above the eye level.

In addition, the window portion 17 may have a light control function capable of changing transparency. In this case, the window portion 17 can be made opaque at the time of confirmation of the magnified image acquired by the imaging unit 20, and can be made transparent at the time of confirmation of surroundings, for example, a search for the instrument 103. The remote controller 40 to be described below may be provided with an operation button used to change the transparency of the window portion 17.

From the viewpoint of visibility of the monitor 30, most of the front surface portion 11a and the upper surface portion 11c of the device body 11 are preferably made of an opaque material. For example, a transparent portion such as the window portion 17 or an opening portion may be provided on the lower surface portion 11d so that the hand can be seen.

The medical magnifier device 10 may have a configuration capable of performing a heat sterilization treatment. In the heat sterilization treatment, for example, the medical magnifier device 10 is exposed to a high-temperature steam environment. For this reason, the medical magnifier device 10 to be subjected to the heat sterilization treatment preferably has a structure in which the imaging unit 20 can be separated from the device body 11, that is, a structure in which the imaging unit 20 is detachable from the device body 11. In addition, the device body 11 and the belt 60 are made of a resin having high formability and heat resistance such as an acrylic resin, polycarbonate, polyester, or polyamide. The belt 60 may be made of leather.

As described above, the imaging unit 20 includes the zoom lens 22, and has a function of performing digital conversion on the magnified image acquired through the zoom lens 22. The imaging unit 20 includes the main body 21 that incorporates an imaging element such as a CCD or a CMOS, as in a digital camera or a camera phone. The zoom lens 22 is installed in front of the main body 21, light passing through the zoom lens 22 is incident on the imaging element of the main body 21 and is converted into a digital signal, and the converted digital signal is displayed on the monitor 30. The main body 21 is provided with, for example, an image processing engine and a storage portion that stores data of the digitally converted magnified image.

The imaging unit 20 may be configured to manually adjust a focus, but preferably has an autofocus function. In this case, the main body 21 incorporates an autofocus sensor, a motor, a control microcomputer, and the like. The main body 21 may be provided with a mechanical shutter for taking a still image.

The zoom lens 22 is a lens used to acquire a magnified image of an affected area, and has an optical zoom function. By the optical zoom lens 22, a clear magnified image of the affected area can be obtained. The magnification of the zoom lens 22 is set in an appropriate range depending on a use purpose; for example, the magnification of the zoom lens is set to 5 times to 40 times, and preferably 5 times to 20 times in the use purpose of a medical examination in obstetrics and gynecology. In a magnifier device dedicated to the medical examination in obstetrics and gynecology, the magnification may be set in a narrow range such as 10 times to 15 times. Furthermore, the imaging unit 20 may have a digital zoom function.

The zoom lens 22 is preferably provided at a position corresponding to a point between the left and right eyes of the user 100, on the front surface portion 11a of the device body 11. In the present embodiment, the device body 11 has a bilaterally symmetrical shape, and the zoom lens 22 is provided at the center, the left side, or the right side in the front surface portion 11a. In order to obtain images corresponding to the monitors 30R and 30L, two zoom lenses 22 can be provided, but one zoom lens 22 is preferably provided in consideration of the lightness of the device.

The monitor 30 includes the right-eye monitor 30R and the left-eye monitor 30L, and is a so-called dual monitor in which a right-eye image is displayed on the monitor 30R and a left-eye image is displayed on the monitor 30L. In the present embodiment, the monitors 30R and 30L are installed on the surface facing the rear of the main body 21, and the respective monitors are isolated from each other by the partition wall portion 15. For example, a liquid crystal monitor or an organic EL monitor is applied to the monitor 30.

For example, the magnified image acquired by the zoom lens 22 and the imaging element is processed for the monitor 30R and the monitor 30L by the image processing engine of the imaging unit 20, and thus the magnified image of the affected area displayed on the monitor 30 is generated.

The medical magnifier device 10 preferably further includes a light 23 provided in front of the device body 11. For example, an LED is applied to the light 23. A separate light may be used when it is necessary to illuminate the affected area brightly, but the mounting of the light 23 on the device body 11 makes the use of the medical magnifier device 10 easier. The light 23 is preferably provided above the zoom lens 22, on the front surface portion 11a of the device body 11.

The medical magnifier device 10 preferably further includes a remote controller 40 (hereinafter, referred to as “remocon 40”) that is wirelessly connected to the imaging unit 20. The remocon 40 includes at least a magnification adjustment button 44 used to operate the magnification of the zoom lens 22. An operation portion may be provided in the device body 11 to perform on/off of the imaging unit 20 and the monitor 30 and adjust the magnification of the zoom lens 22, but the operability is improved by use of the remocon 40. Furthermore, the medical magnifier device 10 may have a function of detecting the line of sight of the user 100 and executing various operations.

The remocon 40 includes a remocon body 41 that is formed thin and long to be easily gripped. A plurality of operation buttons including the magnification adjustment button 44 are provided on one surface of the remocon body 41. Examples of the operation portions include a power switch 42 used to turn on and off the imaging unit 20 and the monitor 30, a light switch 43 used to turn on and off the light 23, a shutter button 45 used to acquire a still image, and a recording button 46 used to acquire a moving image.

The medical magnifier device 10 preferably includes a storage portion that stores the magnified image digitally converted by the imaging unit 20. In the present embodiment, the storage portion is configured by a memory card 52 accommodated in a memory card slot 50. The memory card slot 50 is provided, for example, in the imaging unit 20, and is configured such that the memory card 52 can be inserted from the lower surface portion 11d of the device body 11. The data of the magnified image of the affected area acquired by the imaging unit 20 is retained in the memory card 52. The medical magnifier device 10 may include a storage portion configured by a hard disk (HDD) or the like.

The medical magnifier device 10 may include a power line used to supply power to the imaging unit 20 and the monitor 30, but is preferably driven by a battery 53 because a cord such as the power line interferes with the medical practice. A battery slot 51 is provided, for example, in the imaging unit 20 and is configured such that the battery 53 can be inserted from the lower surface portion 11d of the device body 11. In addition, the imaging unit 20 is provided with one or a plurality of cable jacks 54 into which cables used to connect to a personal computer, a printer, an external monitor, and the like are inserted. When the cable jack 54 is provided, image data and moving image data can be transmitted to the personal computer and the like via the cable.

With reference to FIG. 5, a description will be given herein with respect to a method of performing an medical examination in obstetrics and gynecology, in particular, obstetrics and gynecology cancer screening (thorough checkup of uterine cancer screening) using the medical magnifier device 10 including the configuration described above, and an operational effect of the medical magnifier device 10 will be described in detail.

In the obstetrics and gynecology, a checkup called colposcopy/colposcope diagnosis has been carried out using a colposcope to confirm the presence or absence of a lesion (inflammation, tumor) and the extent of the lesion. In this checkup, a dedicated instrument is inserted into the vagina, and the cervix and the vaginal wall are magnified several times to several tens of times with a colposcope for observation. In addition, acetic acid application diagnosis is also performed which involves, while looking into the colposcope, lightly pressing a cotton ball soaked in an acetic acid solution against the cervix and the like and observing the change in the lesion (observing the change of the lesion due to clouding by acetic acid), and a biopsy is also performed as needed. Further, hemostasis is also performed when there is bleeding.

As described above, the conventional colposcope has a problem that it is large and not so easy to use. On the other hand, according to the medical magnifier device 10 having the above-described configuration, it is possible to perform a simpler and stress-free medical examination while ensuring medical examination accuracy equal to or higher than when the colposcope is used.

As illustrated in FIG. 5, the medical magnifier device 10 is fixed to a head 101 of the user 100 (hereinafter, referred to as “doctor”) using the belt 60 and the auxiliary belt 61. The medical magnifier device 10 is mounted in a state where the face pad 12 comes into contact with the face 102 and the device body 11 covers the left and right eyes to fit with the face 102. Then, the doctor mounted with the medical magnifier device 10 observes the cervix and the vaginal wall by adjusting the zoom lens 22 to an appropriate magnification in a state of turning the zoom lens 22 toward the patient.

In the medical examination in obstetrics and gynecology using the medical magnifier device 10, since the device is fixed to the head 101 of the doctor, there is no need to move the base portion, unlike the colposcope, and an obstacle such as an arm does not exist between the doctor and the patient. Since the focus of the zoom lens 22 is adjusted by autofocus and can also be adjusted by movement of the head 101, there is no need to perform focusing by moving the base portion, unlike the colposcope. When the colposcope is used, there are disadvantages that the arm between the doctor and the patient interferes with the medical practice and focusing also needs to be performed again by contact with the arm.

In the medical examination in obstetrics and gynecology using medical magnifier device 10, as in the case of using the colposcope, the instrument 103 is inserted into the vagina to widen the vagina, and the cervix and the vaginal wall are observed with a magnification of 10 times to 15 times, for example. In the medical magnifier device 10, since the monitors 30R and 30L are disposed in front of the left and right eyes to display the magnified image of the affected area, the magnified image can be easily observed and the medical examination can be performed with good accuracy. Since the magnified images of the affected area displayed on the monitors 30R and 30L are acquired by the optical zoom lens 22 and are digitally converted by the imaging element, the images are bright and clear. In addition, a higher-quality magnified image can be easily obtained using the light 23.

Since the medical magnifier device 10 is firmly fixed to the head 101 and fits with the face 102, the medical practice using both hands can be performed smoothly, and the user hardly get tired even after long time use. Since the device body 11 is provided with the window portions 17 that allows the left and right sides to be visually recognized, the position of the instrument 103 can be confirmed through the window portions 17, for example, and the instrument 103 can be handled without removal of the device one by one. Since the window portions 17 are provided only on both the left and right sides of the device body 11 or on both the left and right sides and in the vicinity thereof, the workability can be improved without impairing the visibility of the monitor 30.

In the medical magnifier device 10, the front can be seen by the zoom lens 22. However, the zoom lens 22 is used to obtain the magnified image, and thus is not suitable for confirming the surrounding situations. Although it is also considered to provide a lens in addition to the zoom lens 22, the weight of the device becomes heavy and switching of the image displayed on the monitor 30 is also necessary in this case.

The medical magnifier device 10 can be easily operated using the remocon 40. For example, the magnification of the zoom lens 22 can be adjusted by the operation of the magnification adjustment button 44. In addition, the still image data of the affected area can be acquired by the operation of the shutter button 45, and the moving image data can also be acquired by the operation of the recording button 46. The still image data and the moving image data are retained in the memory card 52.

As described above, the medical magnifier device 10 is more compact and easier to use compared with the colposcope. According to the medical magnifier device 10, it is possible to perform the medical examination with accuracy equal to or higher than that of the colposcope. The medical magnifier device 10 can be used for the obstetrics and gynecology cancer screening, and is suitable for the thorough checkup of the uterine cancer screening. The medical magnifier device 10 serves as a magnifier device for thorough checkup of uterine cancer screening, for example, and has the excellent effect as described above that is not found in the prior art.

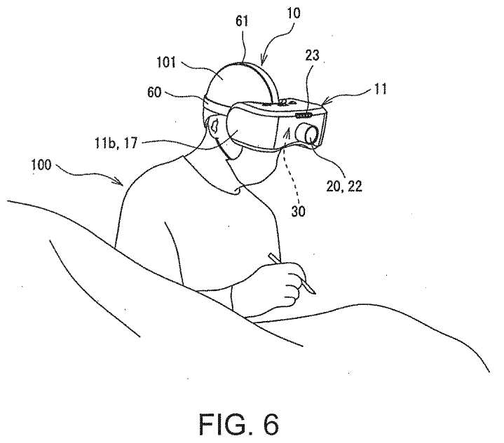

As illustrated in FIG. 6, the medical magnifier device 10 can also be used for surgery. Similarly to the colposcope, a conventional magnifier device (microscope) used for the surgery is large and not so easy to use. In addition, the surgery for a long time while looking through the microscope is an operation having an extremely high fatigue degree. When the surgery is performed using the medical magnifier device 10, the fatigue of the doctor can be reduced and the accuracy of the surgery can also be improved.

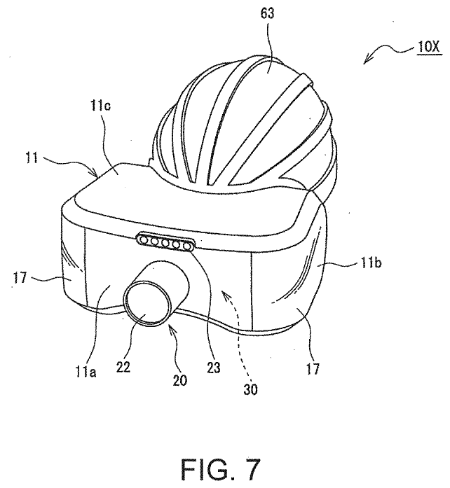

The embodiment described above can be changed in design as appropriate in the range without departing from the object of the present invention. For example, as in a medical magnifier device 10X illustrated in FIG. 7, a helmet-type support portion 63 may be included as the fixing member to cover widely the entire head. The support portion 63 covers the entire head including the front, rear, side, and top of the head, and has a helmet-type shape that is curved along the shape of the head. In the medical magnifier device 10X, the device body 11 is attached to a front end of the support portion 63. Since the device body 11 can be supported with the entire head by the support portion 63, a favorable mounting property is obtained even when the weight of the device body 11 becomes heavy.

### REFERENCE SIGNS LIST

10: medical magnifier device; 11: device body; 11a: front surface portion; 11b: lateral surface portion; 11c: upper surface portion; 11d: lower surface portion; 12: face pad; 13, 13R, 13L: eyepiece; 15: partition wall portion; 16: recess portion; 17: window portion; 18: inter-eyepiece distance adjusting portion; 19R, 19L: eyepiece focus adjusting portion; 20: imaging unit; 21: main body; 22: zoom lens; 23: light; 30, 30R, 30L: monitor; 40: remote controller; 41: remocon body; 42: power switch; 43: light switch; 44: magnification adjustment button; 45: shutter button; 46: recording button; 50: memory card slot; 51: battery slot; 52: memory card; 53: battery; 54: cable jack; 60: belt; 61: auxiliary belt; 63: support portion; 100: user; 101: head; 102: face; 103: instrument

1. A medical magnifier device of a head-mounted type, the medical magnifier device comprising:
a device body that includes a face pad coming into contact with a face of a user and covers left and right eyes of the user;
a fixing member used to fix the device body to a head of the user;
an imaging unit that includes a zoom lens provided in front of the device body and performs digital conversion on a magnified image acquired through the zoom lens; and
at least one monitor that is provided in front of the left and right eyes of the user at the inside of the device body and display the magnified image digitally converted by the imaging unit.

2. The medical magnifier device according to claim 1, further comprising:
a light that is provided in front of the device body to be located above the zoom lens.

3. The medical magnifier device according to claim 1, wherein the device is used for obstetrics and gynecology cancer screening.

4. The medical magnifier device according to claim 1, wherein the device body includes window portions that are provided at both left and right sides with the imaging unit interposed therebetween and allow the user to visually recognize left and right sides.

5. The medical magnifier device according to claim 1, further comprising:
a remote controller that is wirelessly connected to the imaging unit and used to operate a magnification of the zoom lens.

6. The medical magnifier device according to claim 1, wherein the zoom lens is provided in front of the device body to be located at a position corresponding to a point between the left and right eyes of the users.
